(19) United States
(12) Patent Application Publication
(10) Pub. No.: US 2014/0068839 A1
(43) Pub. Date: Mar. 13, 2014

(54) DIAPER SECUREMENT ACCESSORY

(71) Applicants: Matthew Steele, Deltona, FL (US); Donna Steele, Deltona, FL (US)

(72) Inventors: Matthew Steele, Deltona, FL (US); Donna Steele, Deltona, FL (US)

(21) Appl. No.: 13/776,093

(22) Filed: Feb. 25, 2013

Related U.S. Application Data
(60) Provisional application No. 61/604,162, filed on Feb. 28, 2012.

(51) Int. Cl. A61F 13/84

(52) U.S. Cl. CPC A61F 13/84
USPC 2/338

(57) ABSTRACT

A diaper securement accessory is disclosed. The accessory includes a flexible length of material that is adapted to wrap around a diaper. Securement snaps of the accessory are configured to snap into the exposed snap sockets on the front of the diaper. A first free end of the accessory is adapted to overlap an opposing free end and secured to each other using snaps and snap sockets around the back side of the diaper and beyond the toddler's reach. When the diaper securement accessory is firmly secured to the diaper, the toddler is unable to slide the accessory up or down in an effort to take off the diaper. In particular, the right tab and left tab cannot be pulled off the diaper when the accessory is in place.

### I. CROSS REFERENCE TO RELATED APPLICATIONS

This application claims the benefit of U.S. Provisional Application No. 61/604,162 filed Feb. 28, 2012. The disclosure of the provisional application is incorporated herein by reference.

### II. FIELD

The present disclosure is generally related to a diaper securement accessory.

### III. DESCRIPTION OF RELATED ART

Diapers for children may be a disposable type or a reusable cloth type of diaper. Modem day diapers generally have at least two types of material where one type of absorbent material is sandwiched between a comfortable outer layer of material that touches the skin. Diapers are placed on a child around the waist and legs and are adapted to hold bodily fluids and waste.

The outer layer of material is generally made of soft and non-irritating material and may cotton fibers or polyester or polypropylene. In disposable diapers, the absorbent layer is usually formed of cellulose fibers, often comprising polymers capable of forming a gel in contact with liquids. Cloth diapers may only have machine washable natural type of materials.

Although diapers come in many sizes and fashions, most all include some type of closing tabs or ends in the waistband. The diaper is placed in an open position so that the child's bottom is placed thereon so that the waist tabs can be pulled around to the front and removably secured to the front of the diaper using snaps, Velcro, or some other type of securement means. A shortcoming of the prior art is that the tabs are in easy reach of a child so that a child can release the tabs and take off the diaper. This is problematic in the situation where the diaper may be full of waste and leading to an unsanitary condition.

Accordingly, what is needed in the art is a diaper securement accessory that is easy to use and prevents a child from removing his or her diaper.

However, in view of the prior art at the time the present invention was made, it was not obvious to those of ordinary skill in the pertinent art how the identified needs could be fulfilled.

### IV. SUMMARY

In a particular embodiment, a diaper securement accessory is disclosed. The accessory includes a flexible length of material that is adapted to wrap around the diaper. Securement snaps of the accessory are configured on the accessory so that they may be used to snap into the exposed snap sockets on the front of the diaper. In use, a first free end of the accessory overlaps an opposing free end at the back of the diaper and is secured using snaps and snap sockets to secure the accessory to the diaper. The accessory is adapted to be used with a wide variety of existing diapers that have snaps and in particular with cloth style diapers. When the diaper securement accessory is firmly secured to the diaper, the toddler is unable to slide the accessory up or down off the diaper as the right tab and left tab cannot be moved when the accessory is in place. In addition, the toddler cannot remove the accessory as it is secured together at the back of the diaper and behind the toddler's back out of reach.

In an alternative embodiment, the accessory is secured to the diaper using hook and loop material or other alternative securement means such a removable adhesive or hook and loop (Aplix, e.g.). The removable adhesive or hook and loop material is disposed on the inner surface of the accessory and configured to be secured to the front of the diaper. The free ends of the accessory are similar to the preferred embodiment and use snaps to hold together at the back of the diaper and beyond the toddler's reach.

Other aspects, advantages, and features of the present disclosure will become apparent after review of the entire application, including the following sections: Brief Description of the Drawings and Detailed Description.

### V. BRIEF DESCRIPTION OF THE DRAWINGS

FIG. 1 is a front view of a diaper secured around a toddler;

FIG. 2 is a rear view of a diaper securement accessory;

FIG. 3 is a front view of the diaper securement accessory;

FIG. 4 is a front view of the diaper securement accessory secured around the toddler's diaper using the snaps on a front of the diaper;

FIG. 5 is a rear view of the diaper securement accessory with ends of the accessory snapped together;

FIG. 6 is a rear view of an alternative embodiment of the diaper securement accessory; and

FIG. 7 is a front view of the alternative embodiment of the diaper securement accessory shown in FIG. 6.

### VI. DETAILED DESCRIPTION

A toddler's diaper 100 is illustrated in FIG. 1 as it is typically secured around a toddler's legs 101. The diaper 100 shown is a cloth diaper that typically includes a cover that has a few columns of snap sockets 108 disposed on the front. A right tab 102 of the diaper 100 is folded from the back to the front of the diaper and secured using snaps 106 and snap sockets 108. The left tab 104 is secured similarly using snaps 106 and the snap sockets 108. Depending on the size of the toddler, the right tab 102 and the left tab 104 can be secured to the appropriate column of snap sockets 108.

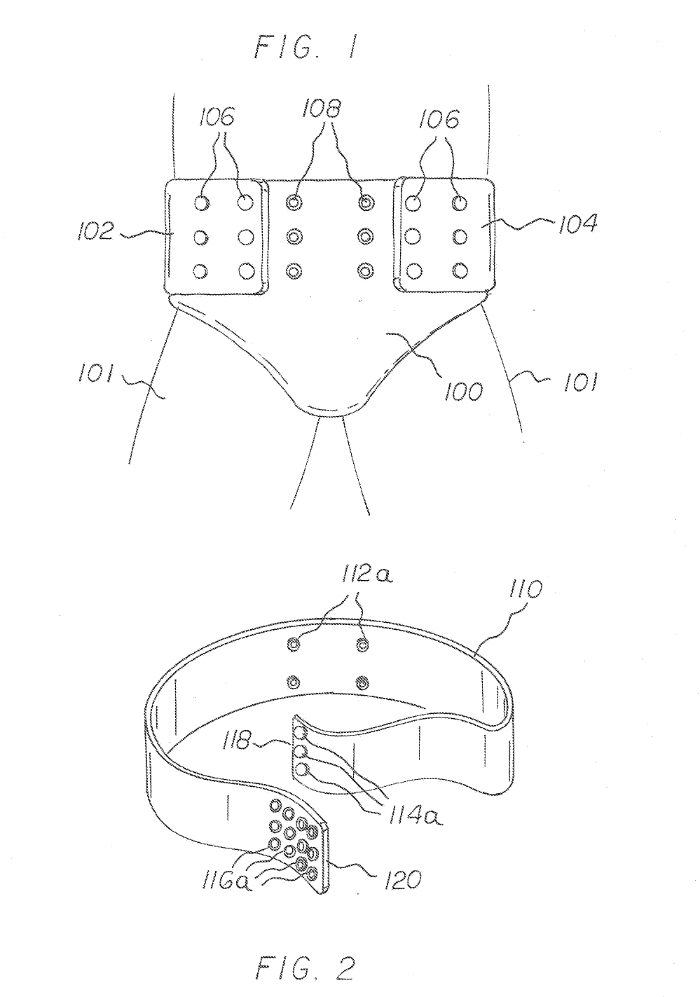

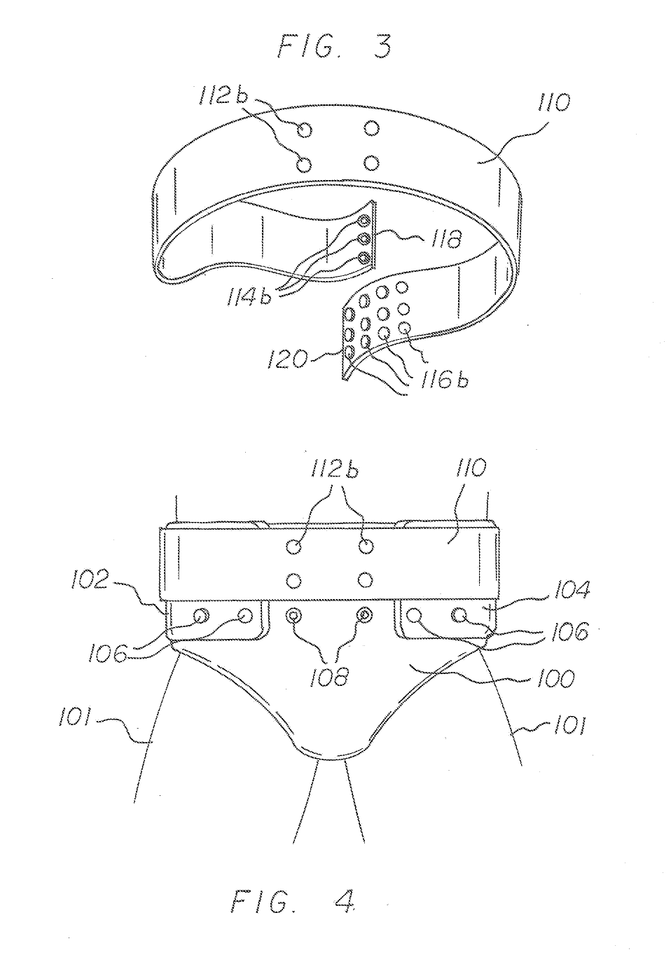

A first particular illustrative embodiment of a diaper securement accessory 110 is disclosed in FIG. 2. The accessory 110 includes a flexible length of material that is adapted to wrap around the diaper 100. Securement snap studs 112a of the accessory 110 are configured on the accessory so that they may be used to snap into the exposed snap sockets 108 on the front of the diaper 100. When the diaper securement accessory 110 is firmly secured to the diaper 100, the toddler is unable to slide the accessory 110 up or down off the diaper 110. In particular, the right tab 102 and left tab 104 cannot be pulled off the diaper 100 when the accessory 110 is in place. A first free end 118 of the accessory 110 is adapted to overlap an opposing free end 120 and secured using snaps and snap sockets 116a to secure the accessory 110 around the diaper 100. Snap covers 114a are shown in FIG. 2, where an opposing side of each snap cover 114a is a snap stud 114b as shown in FIG. 3 described below.

Referring now to FIG. 3, the securement snaps (and snap covers 112b) of the accessory 110 are generally disposed about the center lengthwise of the accessory 110. The inner surface of the accessory 110 includes snap studs 114b that are used to secure free end 118 to the opposing free end 120. The snap covers 116b for the snap studs 116a include a smooth surface to prevent snagging on the diaper 100 when the accessory is put on the toddler.

The diaper securement accessory 110 is shown secured, in FIG. 4, to the diaper 100. The snap covers 112b are visible where the snap studs 112 are snapped into the snap sockets 108 of the diaper 100. The free ends of the accessory 110 are shown overlapping and secured together in FIG. 5. The accessory 110 is adjustable in length by selecting the desired column of snap sockets 116a to snap into to make the accessory cinched tighter or looser as needed.

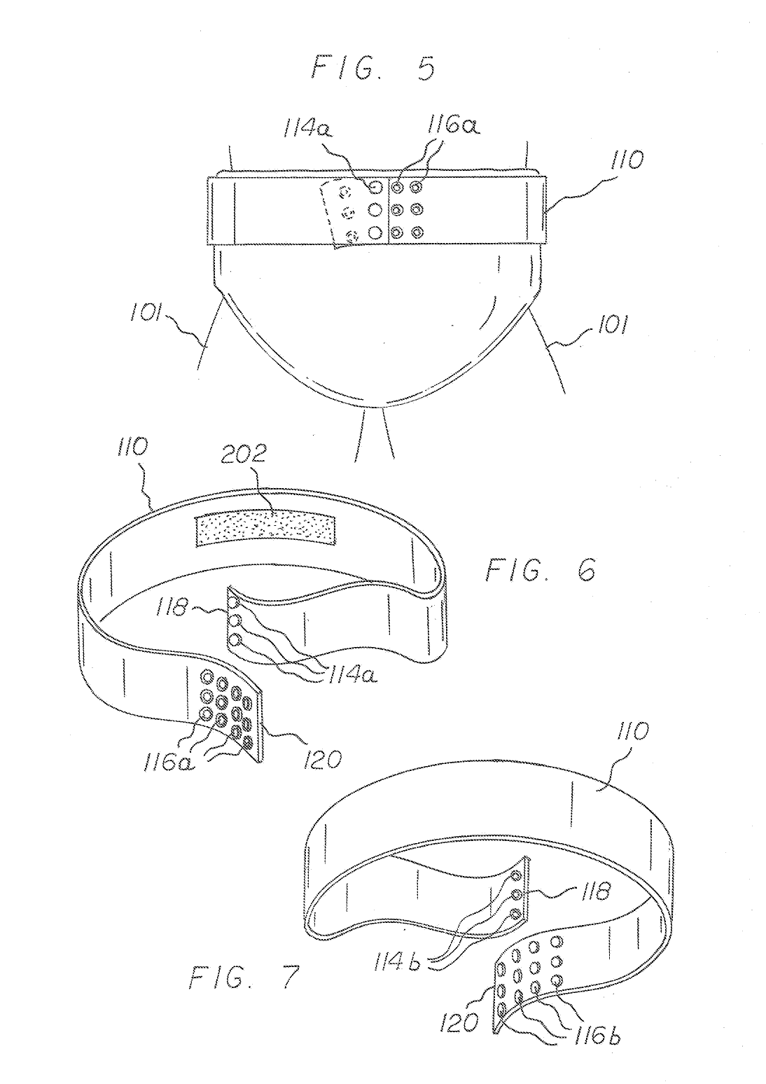

In an alternative embodiment of FIGS. 6 and 7, the accessory 110 is secured to the diaper using hook and loop material 202 or other alternative securement means (Aplix, e.g.). The hook and loop material is disposed on the inner surface of the accessory 110 and configured to be secured to the front of the diaper 100. The free ends 118, 120 of the accessory 110 are similar to the preferred embodiment and use snaps to hold together at the back of the diaper and out of reach of the toddler.

The illustrations of the embodiments described herein are intended to provide a general understanding of the structure of the various embodiments. The illustrations are not intended to serve as a complete description of all of the elements and features of apparatus and systems that utilize the structures or methods described herein. Many other embodiments may be apparent to those of skill in the art upon reviewing the disclosure. Other embodiments may be utilized and derived from the disclosure, such that structural and logical substitutions and changes may be made without departing from the scope of the disclosure. Accordingly, the disclosure and the figures are to be regarded as illustrative rather than restrictive.

One or more embodiments of the disclosure may be referred to herein, individually and/or collectively, by the term “invention” merely for convenience and without intending to voluntarily limit the scope of this application to any particular invention or inventive concept. Moreover, although specific embodiments have been illustrated and described herein, it should be appreciated that any subsequent arrangement designed to achieve the same or similar purpose may be substituted for the specific embodiments shown. This disclosure is intended to cover any and all subsequent adaptations or variations of various embodiments. Combinations of the above embodiments, and other embodiments not specifically described herein, will be apparent to those of skill in the art upon reviewing the description.

What is claimed is:

1. A diaper securement accessory, the accessory comprising:
a flexible length of material configured to wrap around a diaper;
a plurality of securement snap studs on an interior surface of the flexible length of material configured to snap into exposed snap sockets on a front of the diaper; and
a first free end of the length of material configured to overlap an opposing free end at a rear of the diaper and snap together to secure the accessory around the diaper.

2. The diaper securement accessory of claim 1, further comprising a plurality of snap studs on the free end and a plurality of snap sockets on the opposing free end orientated in rows and columns

3. The diaper securement accessory of claim 2, wherein the diaper securement accessory is configured to cover tabs securing the diaper to a child's waist.

4. The diaper securement accessory of claim 3, wherein the diaper is a cloth diaper.

5. The diaper securement accessory of claim 4, wherein the securement snap studs on the interior surface are configured in rows and columns to adjust the accessory to align the securement snap studs with the desired exposed snap sockets on the front of the diaper.

6. The diaper securement accessory of claim 5, wherein the diaper further comprising a cover.

7. A diaper securement accessory, the accessory comprising:
a flexible length of material configured to wrap around a diaper;
a removable adhesive on an interior surface of the flexible length of material configured to hold the accessory to a front of the diaper; and
a first free end of the length of material configured to overlap an opposing free end at a rear of the diaper to secure the accessory around the diaper.

8. The diaper securement accessory of claim 7, further comprising a plurality of snap studs on the free end and a plurality of snap sockets on the opposing free end orientated in rows and columns

9. The diaper securement accessory of claim 7, wherein the removable adhesive on the interior surface is configured to reposition the accessory on the front of the diaper.

10. The diaper securement accessory of claim 7, wherein the diaper securement accessory is configured to cover tabs securing the diaper to a child's waist.
